(19) United States
(12) Patent Application Publication
(10) Pub. No.: US 2003/0211976 A1
(43) Pub. Date: Nov. 13, 2003

(54) Polyamino acid-based particle insulin formulation

(76) Inventors: Kasper Huus Andreasen, Kobenhavn V (DK); Lone Kimer, Farum (DK)

Correspondence Address: Reza Green, Esq., Novo Nordisk Pharmaceuticals, Inc., 100 College Road West, Princeton, NJ, 08540, US

(21) Appl. No.: 10/383,917

(22) Filed: Mar. 7, 2003

Related U.S. Application Data
(60) Provisional application No. 60/363,135, filed on Mar. 8, 2002.

(30) Foreign Application Priority Data
Mar. 7, 2002 (DK) PA 2002 00350

(51) Int. Cl. A61K 38/28; A61L 9/04; A61K 9/14

(52) U.S. Cl. 514/3; 424/46

(57) ABSTRACT

This invention relates to dual-release formulations of insulin comprising polyamino acid particles and insulin, and a method of preparing such formulations

### CROSS-REFERENCE TO RELATED APPLICATIONS

This application claims priority under 35 U.S.C. 119 of Danish application no. PA 2002 00350 filed Mar. 7, 2002 and U.S. application No. 60/353,135 filed Mar. 8, 2002, the contents of which are fully incorporated herein by reference.

### FIELD OF THE INVENTION

This invention relates to dual-release formulations of insulin comprising polyamino acid particles and insulin, and a method of preparing such formulations.

### BACKGROUND OF THE INVENTION

Diabetes is a general term for disorders in man having excessive urine excretion as in diabetes mellitus and diabetes insipidus. Diabetes mellitus is a metabolic disorder in which the ability to utilize glucose is more or less completely lost. About 2% of all people suffer from diabetes.

Since the introduction of insulin in the 1920's, continuous strides have been made to improve the treatment of diabetes mellitus. To help avoid extreme glycemia levels, diabetic patients often practice multiple injection therapy, whereby insulin is administered with each meal.

In the treatment of diabetes mellitus, many varieties of insulin preparations have been suggested and used, such as regular insulin, Semilente® insulin, isophane insulin, insulin zinc suspensions, protamine zinc insulin, and Ultralente® insulin. As diabetic patients are treated with insulin for several decades, there is a major need for safe and life quality improving insulin preparations. Some of the commercially available insulin preparations are characterized by a fast onset of action and other preparations have a relatively slow onset but show a more or less prolonged action. Fast acting insulin preparations are usually solutions of insulin, while retarded acting insulin preparations can be suspensions containing insulin in crystalline and/or amorphous form precipitated by addition of zinc salts alone or by addition of protamine or by a combination of both. In addition, some patients are using preparations having both a fast onset of action and a more prolonged action. Such a preparation may be an insulin solution wherein protamine insulin crystals are suspended. Some patients do themselves prepare the final preparation by mixing an insulin solution with a suspension preparation in the ratio desired by the patient in question.

Protracted insulin compositions are well known in the art. Thus, one main type of protracted insulin compositions comprises injectable aqueous suspensions of insulin crystals or amorphous insulin. In these compositions, the insulin compounds utilised typically are protamine insulin, zinc insulin or protamine zinc insulin.

Certain drawbacks are associated with the use of insulin suspensions. Thus, in order to secure an accurate dosing, the insulin particles must be suspended homogeneously by gentle shaking before a defined volume of the suspension is withdrawn from a vial or expelled from a cartridge. Also, for the storage of insulin suspensions, the temperature must be kept within more narrow limits than for insulin solutions in order to avoid lump formation or coagulation.

Human insulin consists of two polypeptide chains, the so-called A and B chains which contain 21 and 30 amino acids, respectively. The A and B chains are interconnected by two cystine disulphide bridges. Insulin from most other species has a similar construction, but may not contain the same amino acids at the positions corresponding in the chains as in human insulin.

The development of genetic engineering has made it possible easily to prepare a great variety of insulin compounds being analogous to human insulin. In these insulin analogues, one or more of the amino acids have been substituted with other amino acids which can be coded for by the nucleotide sequences. As human insulin, as explained above, contains 51 amino acid residues, it is obvious that a large number of insulin analogues are possible and, in fact, a great variety of analogues with interesting properties have been prepared. In human insulin solutions with a concentration of interest for injection preparations, the insulin molecule is present in associated form as a hexamer (Brange et al. Diabetes Care 13, (1990), 923-954). After subcutaneous injection, it is believed that the rate of absorption by the blood stream is dependent of the size of the molecule, and it has been found that insulin analogues with amino acid substitutions which counteract or inhibit this hexamer formation have an unusual fast onset of action (Brange et al.: Ibid). This is of great therapeutic value for the diabetic patient.

Pharmaceutical preparations which are based on analogues of human insulin have e.g. been presented by Heinemann et al., Lutterman et al. and Wiefels et al. at the “Frontiers in Insulin Pharmacology” International Symposium in Hamburg, 1992.

Furthermore, U.S. Pat. No. 5,474,978 discloses a rapid acting parenteral formulation comprising a human insulin analogue hexamer complex consisting of six monomeric insulin analogues, zinc ions and at least three molecules of a phenolic derivative.

Normally, insulin preparations are administered by subcutaneous injection. What is important for the patient, is the action profile of the insulin preparation which is the action of insulin on the glucose metabolism as a function of the time from the injection. In this profile, inter alia, the time for the onset, the maximum value and the total duration of action are important. A variety of insulin preparations with different action profiles are desired and requested by the patients. One patient may, on the same day, use insulin preparations with very different action profiles. The action profile requested is, for example, depending on the time of the day and the amount and composition of any meal eaten by the patient.

Equally important for the patient is the chemical stability of the insulin preparations, especially due to the abundant use of pen-like injection devices such as devices which contain Penfill® cartridges, in which an insulin preparation is stored until the entire cartridge is empty. This may last for at least 1 to 2 weeks for devices containing 1.5-3.0 ml cartridges. During storage, covalent chemical changes in the insulin structure occur. This may lead to formation of molecules which are less active and potentially immunogenic such as deamidation products and higher molecular weight transformation products (dimers, polymers, etc.). A comprehensive study on the chemical stability of insulin is given in by Jens Brange in “Stability of Insulin”, Kluwer Academic Publishers, 1994.

One way of attaining an injectable protracted insulin preparation is known from U.S. Pat. No. 5,904,936 to Huille et al. This patent discloses delivery vehicles for active principles comprising nano or micrometer sized particles based on polyamino acids. A method of preparing polyamino acids is disclosed in U.S. Pat. No. 5,780,579.

The disadvantage of the insulin preparations of U.S. Pat. No. 5,904,936 is that they will have a protracted action only. It has now been found that adding polyamino acids in a sub-stoichiometric ratio to the insulin solution will result in a dual-release insulin preparation.

### SUMMARY OF THE INVENTION

The present invention encompasses pharmaceutical preparations comprising particles based on polyamino acids in conjunction with an active ingredient that may be insulin, an insulin analogue, an insulin derivative, or combinations thereof, wherein the stoichiometric ratio between the particles and active ingredient is such that a predetermined proportion of the active ingredient in the preparation is in the soluble fraction. In some embodiments, the ratio between the active ingredient adsorbed to particles and dissolved that is dissolved is in the range from about 80:20 to about 20:80; in other embodiments, the ratio is in the range from about 70:30 to about 30:70 and, in yet other embodiments, the ratio is about 70:30.

In one series of embodiments, The polyamino acids used in practicing the invention (i) are linear with alpha-peptide linkages; (ii) comprise at least two types of recurring amino acids which are identical or different from one another, which may be hydrophobic neutral amino acids (AAN), including, without limitation, Leu, Ile, Val, Ala, Pro, and Phe, and mixtures thereof, or amino acids having an ionisable side chain (AAI) in which at least portion of the AAI amino acid being in ionised form, including, without limitation, Glu, Asp, and mixtures thereof; and (iii) have a weight average molar mass M_{w} of not less than 4000 D.

The polyamino acids include, without limitation, block polyamino acids, for which the ratio AAN/(AAN+AAI) mole ratio is ≧6% and M_{w}≧5500 D, such as, e.g., block polyamino acids for which the ratio AAN/(AAN+AAI) mole ratio is ≧5% and 6500 D≦M_{w}≦200000 D, and statistical polyamino acids, for which the AAN/(AAN+AAI) mole ratio is ≧20% and M_{w}≧10000 D, including, e.g., statistical polyamino acids. In some embodiments, the polyamino acids comprise a single type of comonomer AAN and a single type of comonomer AAI. Preferably, the weight average molar mass M_{w} of the polyamino acids is not less than 5000 D.

Typically, the particles comprise from 0.01% to 25% dry weight of the preparation, such as, e.g., from 0.05% to 10% dry weight. The average particle size is typically between 0.03 and 0.4 μm. In some embodiments, the particles further comprise at least one aggregating agent. In some embodiments, the particles further comprise a hydrophilic block-copolymer of the polyalkylene-glycol type, such as, e.g., polyethylene glycol. The total concentration of polyamino acids is typically not less than 10⁻²% weight/volume, such as, e.g., between 0.05 and 30% weight/volume or 0.5 and 5% weight/volume.

The preservative agents may be one or more of EDTA, bronopol, benzyl alcohol, benzoic acid, phenylmercuric acetate, thimerosal, glycerol (glycerin), imidurea, chlorohexidine, sodium dehydroacetate, o-cresol, m-cresol, p-cresol, chlorocresol, benzyl alcohol, benzalkonium chloride, cetrimide, benzethonium chloride, methylparaben, ethylparaben, propylparaben, or butylparaben. In one embodiment, the preservative agent is one or more phenolic preservatives, including, without limitation, phenol, m-cresol, or a combination of phenol and cresol. The total concentration of the one or more preservative agents may be, without limitation, 20 to 50 mM, such as, e.g., 32 to 48 mM, 36 to 42 mM, or 38 to 40 mM. In one embodiment, the preparation comprises 16 to 24 mM phenol and 16 to 24 mM m-cresol; in another embodiment, 19 to 21 mM phenol and 19 to 21 mM m-cresol.

In some embodiments, the insulin analogue is an analogue of human insulin, including, without limitation (i) an analogue in which position B28 is Asp, Lys, Leu, Val, or Ala and position B29 is Lys or Pro, such as, e.g., an analogue in which position B28 is Asp or Lys, and position B29 is Lys or Pro; or (ii) des(B28-B30), des(B27) or des(B30) human insulin.

In some embodiments, the insulin derivative is a derivative of human insulin having one or more lipophilic substituents, including, without limitation, B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28} Pro^{B29} human insulin, B28-N^{ε}-palmitoyl Lys^{B28} Pro^{B29} human insulin, B30-N^{ε}-myristoyl-Thr_{B29}Lys^{B30} human insulin, B30-N^{ε}-palmitoyl-Thr^{B29}Lys^{B30} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin, and B29-N^{ε}-(ω-carboxyheptadecanoyl) human insulin.

Typically, the concentration of insulin in the preparations of the invention is from 60 to 3000 nmol/ml, such as, e.g., from 240 to 1200 nmol/ml.

The invention also encompasses methods for preparing a pharmaceutical preparation, which are carried out by the steps of (i) mixing a polyamino acid particle solution with a solution comprising an active ingredient selected from the group consisting of insulin, an insulin analogue, an insulin derivative, and combinations of any of the foregoing; (ii) incubating the mixture, and, optionally (iii) adding one or more further constituents, under conditions in which, after the mixing step, the ratio between insulin adsorbed to particles and dissolved insulin is in the range from about 95:5 to about 5:95.

In another aspect, the invention encompasses a pharmaceutical preparation comprising (i) particles based on polyamino acids and (ii) an active ingredient that may be insulin, an insulin analogue, an insulin derivative, or combinations thereof, wherein at least 5% of the active ingredient in the preparation is not absorbed to the particles. The proportion of active ingredient not absorbed to the particles (e.g., free in solution) may be at least 5%, 10%, 25%, 50%, or 75%.

In another aspect, the present invention provides methods for treating diabetes, that are carried out by administering to a patient in need of such treatment an effective amount of the preparations of the invention.

In yet another aspect, the invention provides a method of designing a sustained-release formulation for treating diabetes, which is carried out by the steps of:

(i) providing a plurality of preparations of polyamino acids that form particles when in solution;

(ii) individually mixing the preparations with a plurality of active ingredients, which may be insulin, insulin analogues, insulin derivatives, or mixtures thereof, to form a matrix of test mixtures in which the ratio between active ingredient adsorbed to particles and dissolved active ingredient is in the range from about 95:5 to about 5:95; and

(iii) testing the ability of individual mixtures within the matrix to modulate blood glucose levels in a model system, to identify one or more mixtures that provide a predetermined blood glucose profile.

The method may further comprise

(iv) repeating steps (i)-(iii) until a mixture providing a desired blood glucose profile is identified.

### BRIEF DESCRIPTION OF THE DRAWINGS

FIG. 1 is a graphic representation showing changes in plasma glucose (%) Mean±SEM, after subcutaneous injection in pigs of insulin and 42.5 mg polymer/ml (N=4) or 55 mg polymer/ml (N=5) polyamino acid particles.

FIG. 2 is a graphic representation showing changes in plasma glucose (%) Mean±SEM, after subcutaneous injection of Insulin Aspart and 100 mg polymer/mL polyamino acid particles.

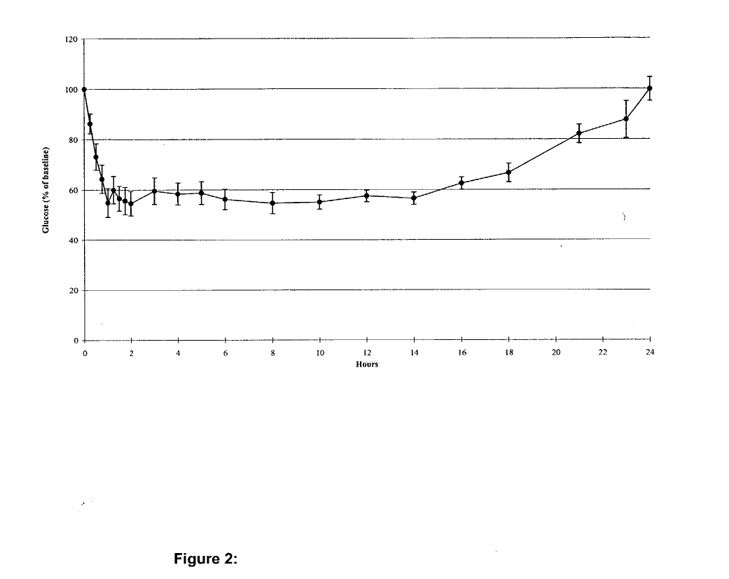

FIG. 3 is graphic representation showing changes in plasma glucose (%) Mean±SEM, after subcutaneous injection of Insulin Aspart and 50 mg polymer/mL or 100 mg polymer/mL polyamino acid particles.

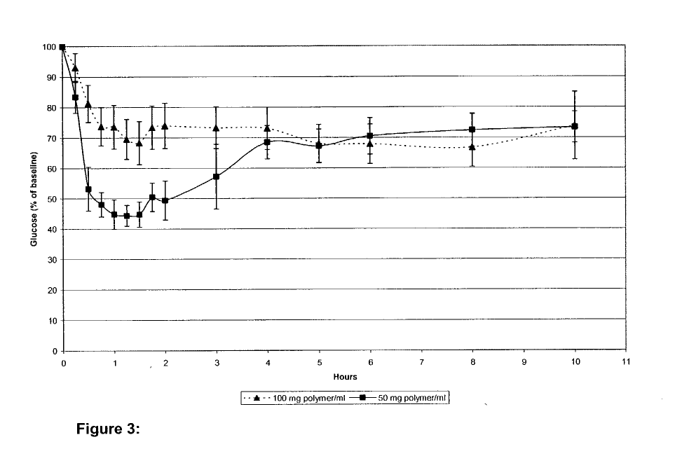

### DETAILED DESCRIPTION OF THE INVENTION

Definitions

The term insulin as used herein refers to human insulin or an analogue thereof or a derivative thereof. However, porcine insulin is also an insulin species that may be employed with the present invention. Preferably, porcine insulin is highly purified naturally produced porcine insulin.

The term human insulin as used herein refers to naturally produced insulin or recombinantly produced. Recombinant human insulin may be produced in any suitable host cell for example the host cells may be bacterial, fungal (including yeast), insect, animal or plant cells.

The term human insulin analogue as used herein refers to human insulin in which one or more amino acids have been deleted and/or replaced by other amino acids, including non-codeable amino acids, or human insulin comprising additional amino acids, i.e. more than 51 amino acids.

The term phenolic preservative as used herein refers to a chemical compound in which a hydroxyl group is bound directly to a benzene or substituted benzene ring. Examples of such compounds include, but are not limited to, phenol, o-cresol, m-cresol and p-cresol.

This invention relates to dual-release formulations of insulin comprising sub-stoichiometric amounts of polyamino acid particles and insulin, and a method of preparing such formulations.

The polyamino acid particles may be prepared according to the disclosure of U.S. Pat. No. 5,904,936, which is hereby incorporated by reference. In one embodiment of the invention, the particles are prepared according to the method described in example 1 of U.S. Pat. No. 5,904,936. In other embodiments of the invention, the particles are prepared according to the methods described in examples 2, 3, 4, 5, 8, 16, 17, or 18 of U.S. Pat. No. 5,904,936. Particles further comprising polyalkylene-glycol are disclosed in WO 02/78677, and may be prepared by a method described e.g. in example 1 or 2 of said reference.

In another embodiment of the invention, the particles are prepared according to the disclosure of WO 00/30618 which is hereby incorporated by reference, e.g. page 9, line 22 to page 10, line 9. In one embodiment of the invention, the particles are prepared according the method described in example 1 of WO 00/30618. In other embodiments of the invention, the particles are prepared according to the methods described in examples 2, 3, or 4 of WO 00/30618.

In another embodiment of the invention, the particles are prepared according to the disclosure of WO 01/37809 which is hereby incorporated by reference, e.g. page 14, line 1 to page 17, line 10. In one embodiment of the invention, the particles are prepared according the method described in example 1 of WO 01/37809. In other embodiments of the invention, the particles are prepared according to the methods described in examples 3, 4, 5, or 6 of WO 01/37809. The particles are based on amphiphilic, linear polyamino acids with alpha-peptide linkages, and comprise at least two different types of recurrent amino acids, i.e. hydrophilic and neutral hydrophobic amino acids, the amino acids within each group being either identical or different. The hydrophilic amino acids are chosen among those with ionisable side chains, with amino acids Glu and Asp in carboxylic or salt form being particularly preferred. The neutral hydrophobic amino acids are chosen from natural neutral amino acids, preferably those belonging to the sub-group comprising Leu, Ile, Val, Ala, Pro, and Phe. The particles are stable at a pH between 4 and 13 in the absence of a surfactant. They have an insulin load factor ranging between 5 and 25% of associated insulin volume relative to the polyamino acid volume. The particles have a mean hydrodynamic diameter between 10 and 150 nm, preferably between 20 and 100 nm. The mean hydrodynamic diameter is measured as described in WO 01/37809 on page 9, line 11 to line 21. The insulin load factor is measured by the method described in WO 01/37809 on page 9, line 23 to page 10, line 7.

The amount of polyamino acid particles in the final pharmaceutical preparation of the present invention is sub-stoichiometric, i.e. present in an amount which is insufficient to adsorb all of the insulin present in the preparation. The result is a pharmaceutical preparation which contains both insulin adsorbed to polyamino acid particles as well as a substantial amount of insulin in dissolved form.

In a further embodiment of the invention two separate formulations are provided to the patient or medical personnel, one containing rapid-acting insulin and another containing insulin adsorbed to polyamino acid particles. Prior to administration the patient or medical personnel mixes the two formulations in a ratio determined to result in the desired action profile.

The formulation moreover comprises insulin. In one embodiment the insulin is selected from the group consisting of human insulin and analogues thereof as well as combinations of these. In one embodiment of the invention the insulin which is adsorbed to the polyamino acid particles is different from the insulin present in dissolved form. In another embodiment the insulin which is adsorbed to the polyamino acid particles is human insulin, while the insulin present in dissolved form is an analogue of human insulin.

In another embodiment the human insulin analogue is selected from the group consisting of

i. An analogue wherein position B28 is Asp, Lys, Leu, Val, or Ala and position B29 is Lys or Pro; and

ii. des(B28-B30), des(B27) or des(B30) human insulin.

In another embodiment the insulin is an analogue of human insulin wherein position B28 is Asp or Lys, and position B29 is Lys or Pro. In another embodiment the insulin is des(B30) human insulin. In another embodiment the insulin is LysB3 GluB29.

In one embodiment of the invention the stoichiometric ratio of dissolved active ingredient to active ingredient adsorbed to polyamino acid particles is from about 5:95 to about 95:5. In another embodiment is from about 20:80 to about 80:20. In another embodiment the ratio particles is from about 30:70 to about 70:30. In another embodiment the ratio is about 30:70.

It will be understood that the stoichiometric ratio of dissolved active ingredient to adsorbed active ingredient may be measured by (i) fractionating the preparation into a particulate fraction and a soluble fraction and (ii) determining the amount of active ingredient in at least one, and preferably both, of the fractions. Both the fractionation and the determination steps may be carried out by any means conventional in the art. For example, fractionation may be achieved by, e.g., centrifugation, filtration, ultrafiltration, precipitation, or combinations thereof. Determination of active ingredient may be carried out by, e.g., radioimmunoassay; measurement of active ingredient that has been previously labelled using tracer amounts of radiolabel, fluorescent label, and the like; bioassay, chromatographic or electrophoretic separation, spectroscopic identification, and the like. Typically, following fractionation, measurement of the active ingredient is performed and compared with the starting (unfractionated) preparation in order to determine the particulate: soluble ratio.

The formulation according to the present invention may furthermore comprise one or more preservative agents.

In one embodiment of the invention the preservative agent is selected from the group consisting of EDTA, bronopol (e.g. in a concentration of 0.5 to 5 mM), benzyl alcohol (e.g. in a concentration of 80 to 300 mM), benzoic acid (e.g. in a concentration of 10 to 100 mM), phenylmercuric acetate, thimerosal, glycerol (glycerin), imidurea, chlorohexidine, sodium dehydroacetate, phenolic preservatives such as phenol, o-cresol, m-cresol, p-cresol, or chlorocresol (the latter e.g. in a concentration of 1 to 20 mM), benzalkonium chloride, cetrimide, benzethonium chloride, methylparaben, ethylparaben, propylparaben, or butylparaben (the total concentration of parabenes being e.g. in the range from 0.02 to 0.3% (W/V), or a combination of one or more of these.

In another embodiment the preservative agent is one or more phenolic preservatives.

In another embodiment the preservative is phenol or m-cresol, or a combination of both.

In another embodiment the total concentration of the one or more phenolic preservatives is 20 to 50 mM.

In another embodiment the total concentration of the one or more phenolic preservatives is 32 to 48 mM.

In another embodiment the total concentration of the one or more phenolic preservatives is 36 to 42 mM.

In another embodiment the total concentration of the one or more phenolic preservatives is 38 to 40 mM.

In another embodiment the concentration of the phenolic preservatives is 16 to 24 mM of phenol and 16 to 24 mM of m-cresol.

In another embodiment the concentration of the phenolic preservatives is 19 to 21 mM of phenol and 19 to 21 mM of m-cresol.

In another embodiment the formulation comprises 30 to 48 mM of m-cresol. In another embodiment the formulation comprises 36 to 42 mM of m-cresol.

The preparations of this invention may contain further constituents such as a zinc salt, for example zinc chloride, an isotonic agent, for example sodium chloride or glycerol, a surfactant, for example poloxamers or polysorbat, and a buffer, for example disodium monohydrogen phosphate, in an aqueous medium. The pH of the preparation may furthermore be adjusted, e.g. to a pH value of from 4 to 8. In another embodiment, the pH is adjusted to a pH value of about 7.3.

The amount of polyamino acid particles added to the pharmaceutical preparation of the invention can be determined in two ways. One is described in U.S. Pat. No. 5,904,936, see e.g. example 14. The amount of insulin adsorbed to the polyamino acid particles is calculated as the difference between added insulin and insulin present in dissolved form after filtration of the preparation. In this way, a person skilled in the art will be able to determine by routine experimentation the amount of polyamino acid particles necessary to adsorb a given amount of insulin. Another method is to carry out experiments in an animal model with preparations containing insulin adsorbed to varying amounts of polyamino acid particles. One model is measurement of the blood glucose lowering effect of the product when administered subcutaneously to pigs. The onset and duration of the blood glucose lowering effect is dependent on the amount of poly amino acid particles relative to the amount of insulin; this ratio determines the amount of free insulin, which provides a fast onset, and the amount of insulin adsorbed to the poly amino acid particles, which provides a prolonged effect. Thus, if a fast on-set is observed, a surplus of insulin is known to have been present in the preparation.

In one aspect of the invention the present pharmaceutical preparation is made available to the patient or medical personnel in the form of vials containing the preparation. In another aspect, the preparation is made available in the form of a cartridge for use in pen injector devices. Such devices may be either disposable or durable. The preparation may also be used in an insulin pump system.

The invention also relates to a method of treating diabetes in a patient comprising administering to said patient a pharmaceutical preparation of the invention.

The pharmaceutical preparation according to the present invention is made in a method which comprises the following steps:

1. Mixing polyamino acid particles with an insulin solution.

2. Incubating

3. Optionally adding one or more further constituents

In one embodiment of the invention the insulin is added in two stages, with an incubation step in between. In a further embodiment the insulin used in the second stage is different from the insulin used in the first stage, e.g. the first stage is carried out with human insulin, and subsequently an analogue of human insulin is added after an incubation step.

In one embodiment of the invention, the concentration of the insulin in the final preparation is from 60 to 3000 nmol/ml. In another embodiment, the concentration of the insulin in the final preparation is from 240 to 1200 nmol/ml. In another embodiment, the incubation is carried out at room temperature. In another embodiment, the incubation has a duration of from 1 to 24 hours, preferably from 6 to 12 hours. In another embodiment, the preservative agent is added after the mixing of polyamino acids and insulin solution. In another embodiment, the preservative agent is added after incubation.

### EXAMPLES

The following examples are intended as non-limiting illustrations of the present invention.

In Vivo Experiments

### Example 1

Polyamino Acid Particles and Human Insulin.

Domestic pigs were injected subcutaneously with preparations containing 42.5, 50.0 or 55.0 mg polymer/mL respectively and 600 nmol/mL human insulin, 21 mM phenol, 21 mM m-cresol and 15 μg Zn²⁺/ml. The pH was adjusted to 7.4 and NaCl was added to achieve isotonic conditions. Blood samples were withdrawn at selected time points for 24 hours. The plasma was separated by centrifugation and plasma glucose values were obtained by standard glucose hexokinase assay.

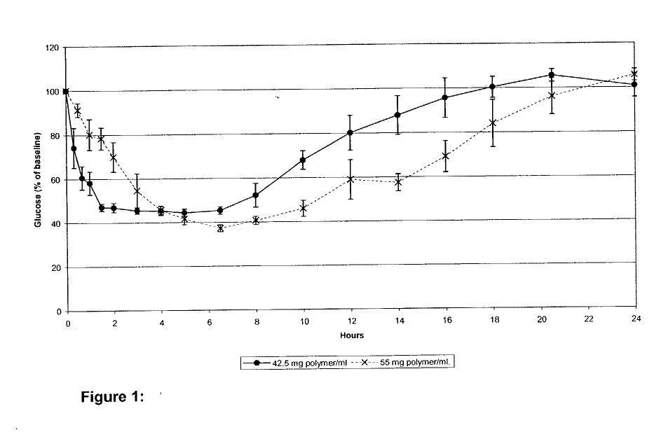

The results are shown in FIG. 1.

### Example 2

Polyamino Acid Particles and Insulin Aspart.

Domestic pigs were injected subcutaneously with preparations containing 100 mg/mL polymer, 600 nmol/mL insulin aspart, 10 mM Na₂HPO₄ and 15 μg Zn²⁺. The pH was adjusted to 7.3 and NaCl was added to achieve isotonic conditions. Blood samples were withdrawn at selected time points for 24 hours. The plasma was separated by centrifugation and plasma glucose values were obtained by standard glucose hexokinase assay.

[image]

### Example 3

Polyamino Acid Particles and Insulin Aspart

Domestic pigs were injected subcutaneously with preparations containing 50 and 100 mg/mL polymer respectively, 600 nmol/mL insulin aspart, 10 mM Na₂HPO₄ and 15 μg Zn²⁺. The pH was adjusted to 7.3 and NaCl was added to achieve isotonic conditions. Blood samples were withdrawn at selected time points for 10 hours. The plasma was separated by centrifugation and plasma glucose values were obtained by standard glucose hexokinase assay.

All patents, patent applications, and literature references referred to herein are hereby incorporated by reference in their entirety.

Many variations of the present invention will suggest themselves to those skilled in the art in light of the above detailed description. Such obvious variations are within the full intended scope of the appended claims.

1. A pharmaceutical preparation comprising
i. Particles based on polyamino acids wherein said polyamino acids
a. are linear with alpha-peptide linkages,
b. Comprise at least two types of recurring amino acids which are identical or different from one another, selected from the group consisting of a hydrophobic neutral amino acid (AAN), and an amino acid having an ionisable side chain (AAI), at least portion of the AAI amino acid being in ionised form, and
c. Have a weight average molar mass M_{w} of not less than 4000 D; and
ii. An active ingredient selected from the group consisting of insulin, an insulin derivative, an insulin analogue, and combinations of any of the foregoing,
wherein the ratio between active ingredient adsorbed to particles and dissolved active ingredient is in the range from about 95:5 to about 5:95.

2. A pharmaceutical preparation according to claim 1 wherein the particles comprise polyamino acids selected form the group consisting of block and statistical polyamino acids, wherein for the block polyamino acids, the ratio AAN/(AAN+AAI) mole ratio is ≧6% and M_{w}≧5500 D, and for the statistical polyamino acids the AAN/(AAN+AAI) mole ratio is ≧20% and M_{w}≧210000 D.

3. A pharmaceutical preparation according to claim 1, wherein the hydrophobic neutral amino acid is selected from the group consisting of Leu, Ile, Val, Ala, Pro, Phe, and mixtures thereof; and the amino acid having an ionisable side chain is selected from the group consisting of Glu, Asp, and mixtures thereof.

4. A pharmaceutical preparation according to claim 1, wherein the average polyamino acid concentration of the particles is from 0.01% to 25% dry weight.

5. A pharmaceutical preparation according to claim 4, wherein the concentration is from 0.05% to 10% dry weight.

6. A pharmaceutical preparation according to claim 1, wherein the average particle size is between 0.03 and 0.4 μm.

7. A pharmaceutical preparation according to claim 1, wherein the weight average molar mass M_{w} of the polyamino acids is not less than 5000 D.

8. A pharmaceutical preparation according to claim 2, wherein for the block polyamino acids, the ratio AAN/(AAN+AAI) mole ratio is ≧5% and 6500 D≦M_{w}≦200000 D, and for the statistical polyamino acids the AAN/(AAN+MI) mole ratio is ≧25% and 20000 D≦M_{w}≦500000 D.

9. A pharmaceutical preparation according to claim 8, wherein for the block polyamino acids, 8000 D≦M_{w}≦200000 D, and for the statistical polyamino acids 20000 D≦M_{w}≦150000 D.

10. A pharmaceutical preparation according to claim 1, wherein the particles further comprise at least one aggregating agent.

11. A pharmaceutical preparation according to claim 1, wherein the particles further comprise a hydrophilic block-copolymer of the polyalkylene-glycol type.

12. A pharmaceutical preparation according to claim 11, wherein the hydrophilic block-copolymer of the polyalkylene-glycol type is polyethylene-glycol.

13. A pharmaceutical preparation according to claim 1, wherein the polyamino acids comprise a single type of comonomer MN and a single type of comonomer Ml.

14. A pharmaceutical preparation according to claim 1, wherein the active ingredient is selected from the group consisting of human insulin and analogues of human insulin.

15. A pharmaceutical preparation according to claim 14, wherein the analogue of human insulin is selected from the group consisting of
iii. An analogue wherein position B28 is Asp, Lys, Leu, Val, or Ala and position B29 is Lys or Pro; and
iv. des(B28-B30), des(B27) or des(B30) human insulin.

16. A pharmaceutical preparation according to claim 15, wherein the analogue of human insulin comprises Asp or Lys at position B28, and Lys or Pro at position B29.

17. A pharmaceutical preparation according to claim 15, wherein the analogue of human insulin comprises Asp at position B28.

18. A pharmaceutical preparation according to claim 15, wherein the insulin analogue is des(B30) human insulin.

19. A pharmaceutical preparation according to claim 1, wherein the ratio between insulin adsorbed to particles and dissolved insulin is in the range from about 80:20 to about 20:80.

20. A pharmaceutical preparation according to claim 19, wherein the ratio between insulin adsorbed to particles and dissolved insulin is in the range from about 70:30 to about 30:70.

21. A pharmaceutical preparation according to claim 20 wherein the ratio between insulin adsorbed to particles and dissolved insulin about 70:30.

22. A method of preparing a pharmaceutical preparation, said method comprising the steps of
1. Mixing a polyamino acid particle solution with a solution comprising an active ingredient selected from the group consisting of insulin, an insulin analogue, an insulin derivative, and combinations of any of the foregoing,
2. Incubating the mixture, and, optionally
3. Adding one or more further constituents,
Wherein, after said mixing step, the ratio between insulin adsorbed to particles and dissolved insulin is in the range from about 95:5 to about 5:95.

23. A method according to claim 22, wherein a preservative agent is added to the preparation after the active ingredient and polyamino acid solutions are mixed.

24. A pharmaceutical preparation comprising (i) particles based on polyamino acids and (ii) an active ingredient selected from the group consisting of insulin, an insulin analogue, an insulin derivative, and combinations of any of the foregoing, wherein at least 5% of the active ingredient in the preparation is not absorbed to the particles.

25. A preparation according to claim 24, wherein at least 5% of the active ingredient in the preparation is not absorbed to the particles.

26. A preparation according to claim 25, wherein at least 10% of the active ingredient in the preparation is not absorbed to the particles

27. A preparation according to claim 26, wherein at least 25% of the active ingredient in the preparation is not absorbed to the particles

28. A preparation according to claim 27, wherein at least 50% of the active ingredient in the preparation is not absorbed to the particles.

29. A preparation according to claim 24, wherein the active ingredient is selected from the group consisting of insulin, an insulin derivative, an insulin analogue, and combinations of any of the foregoing.

30. A preparation according to claim 29, wherein the active ingredient is an insulin analogue.

31. A preparation according to claim 30, wherein said insulin analogue is an analogue of human insulin wherein position B28 is Asp or Lys, and position B29 is Lys or Pro.

32. A preparation according to claim 30, wherein the insulin analogue, is des(B30) human insulin.

33. A preparation according to claim 24, wherein the active ingredient is a combination of human insulin and an analogue of human insulin wherein position B28 is Asp or Lys, and position B29 is Lys or Pro.

34. A preparation according to claim 24, wherein the polyamino acids are:
(i) linear with alpha-peptide linkages,
(ii) Comprise at least two types of recurring amino acids which are identical or different from one another, selected from the group consisting of a hydrophobic neutral amino acid (AAN), and an amino acid having an ionisable side chain (AAI), at least portion of the AAI amino acid being in ionised form, and
(iii) Have a weight average molar mass M_{w} of not less than 4000 D.

35. A method of treating diabetes, said method comprising administering to a patient in need of such treatment an effective amount of a preparation according to claim 1.

36. A method of treating diabetes, said method comprising administering to a patient in need of such treatment an effective amount of a preparation according to claim 24.

37. A method of designing a sustained-release formulation for treating diabetes, said method comprising:
(i) providing a plurality of preparations of polyamino acids that form particles when in solution;
(ii) individually mixing said plurality of preparations with a plurality of active ingredients, wherein said active ingredients are selected from the group consisting of insulin, insulin analogues, insulin derivatives, and mixtures of any of the foregoing, to form a matrix of test mixtures in which the the ratio between active ingredient adsorbed to particles and dissolved active ingredient is in the range from about 95:5 to about 5:95; and
(iii) testing the ability of individual mixtures within the matrix to modulate blood glucose levels in a model system, to identify one or more mixtures that provide a predetermined blood glucose profile;

38. A method according to claim 37, further comprising:
(iv) repeating steps (i)-(iii) until a mixture providing a desired blood glucose profile is identified.
